(19) United States
(12) Patent Application Publication
(10) Pub. No.: US 2009/0287258 A1
(43) Pub. Date: Nov. 19, 2009

(54) Bone fixation device and method

(76) Inventor: Prasad VANNEMREDDY, Shreveport, LA (US)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 12/153,139

(22) Filed: May 14, 2008

(51) Int. Cl. A61B 17/80; A61B 17/56

(52) U.S. Cl. 606/298; 606/280; 606/70

(57) ABSTRACT

A plate for contacting at least two cervical vertebrae of a spine, including a first portion configured to contact the first cervical vertebra of the spine, the first portion including a hole arranged on a longitudinal axis of the plate and configured to receive a screw adapted to be screwed into the anterior tubercle of the first cervical vertebra. The plate also includes a second portion configured to contact at least one of the second and third cervical vertebrae of the spine, the second portion including a pair of holes arranged on an axis substantially perpendicular to the longitudinal axis of the plate, each of the holes in the pair of holes being configured to receive a screw adapted to be screwed into the body of one of the second and third cervical vertebrae.

### BACKGROUND

1. Field

The present invention generally relates to surgical devices and to methods for performing spinal surgery. More particularly, the present invention relates to bone fixation devices and to methods for performing anterior fixation of one or more of the second, third, and fourth cervical vertebrae to the first cervical vertebra (C1).

2. Background

Surgeons sometimes perform fixation of one or more bones or bone portions to treat or alleviate various conditions such as bone fractures, tumors, or other diseases. For example, when performing surgeries such as a cervical discectomy, wherein an intervertebral disc is removed, and a cervical fusion, wherein a bone graft is substituted for a removed intervertebral disc and allowed to fuse with surrounding vertebrae, surgeons may perform fixation of those surrounding vertebrae. Such cervical fixation can substantially improve patient healing and rehabilitation because it minimizes movements of the bone graft relative to the surrounding vertebrae and helps ensure that the proper alignment of the spine is maintained as the patient heals.

Several cervical bone fixation approaches are known in the art. These approaches are generally classified according to whether they involve posterior or anterior spinal surgery and to whether they implicate atlantoaxial or subaxial fixation. Atlantoaxial fixation relates to the first and second cervical vertebrae of the spine (also called the atlas and the axis or, alternatively, C1 and C2), whose role is to allow rotation of the head. Subaxial fixation relates to the five cervical vertebrae below the axis (also called C3, C4, C5, C6, and C7), whose role is to support the atlas and the axis without actually playing a role in the rotation of the head. Additional information about bones, the spine, and other anatomical features can be found in Henry Gray, Gray's Anatomy: Descriptive and Surgical, Fifteenth Edition (T. Pickering Pick & Robert Howden, eds., Barnes & Nobles Books, 1995), the entire disclosure of which is incorporated herein by reference.

Subaxial posterior approaches include wire techniques, lateral mass plate fixation, and lateral mass screw fixation; subaxial anterior approaches include anterior odontoid screw fixation, subaxial anterior cervical plates, and anterior cervical threaded cages; atlantoaxial posterior approaches include posterior C1-C2 wiring, C1-C2 transarticular screw fixation, C1 lateral mass screw fixation, C2 pedicle screw fixation, and C2 laminar screw fixation; and atlantoaxial anterior approaches include anterior C1-C2 intra-articular screw fixation, which involves a C1-C2 lateral mass screw but no plating, and anterior C1-C2 lateral mass plates, which only involves lateral plating far from the midline of the spine. These approaches are discussed in detail in Amir H. Fayyazi & Howard S. An, Anterior/Posterior Cervical Instrumentation, in Complications of Spine Surgery: Treatment and Prevention (Howard S. An & Louis G. Jenis, eds., Lippincott Williams & Wilkins, 2006), pp. 55-75, hereinafter “Fayyazi & An,” the entire disclosure of which is incorporated herein by reference.

These approaches fail to satisfactorily address numerous clinically significant injuries and pathological conditions, such as atlantoaxial injuries, odontoid or otherwise, that may require bone removal and subsequent fixation; atlantoaxial dislocations, which can result from certain atlantoaxial injuries such as “Hangman's fractures” and may require surgery to reduce the extent of any dislocation; and the destruction of the odontoid process and/or pannus tissue that can result from rheumatoid arthritis or from atlantoaxial tumors associated with plasmacytoma and metastatic bone disease.

In particular, subaxial posterior and anterior approaches are not very useful for atlantoaxial injuries because the functions and structures of the atlas and axis are completely different from those of subaxial vertebrae, which generally renders application or generalization of subaxial methods and devices to the atlas and axis inappropriate. Atlantoaxial posterior approaches are problematic because they may require multiple operations, which may increase the likelihood of patient complications and post-operative discomfort, along with the number of days the patient will likely remain hospitalized, thus causing increased health care costs. Atlantoaxial posterior approaches are also problematic because they involve a high risk of injury to important structures like nerve roots and blood vessels, and because they can lead to severe restriction of neck movements along with related neck pain and stiffness due to the additional operative exposure and to the posterior fixation devices.

Finally, atlantoaxial anterior approaches such as anterior C1-C2 intra-articular screw fixation and C1-C2 lateral mass plates are not satisfactory because they do not allow a sufficiently safe surgical fixation and do not offer a fixation likely to remain sufficiently stable for optimal patient healing and rehabilitation, at least in part because they do not perform fixation sufficiently close to the midline of the spine and involve implantation that is too lateral, which increases the risks of infection and inadvertent injury to the patient resulting from surgery. Moreover, the indication for anterior C1-C2 intra-articular screw fixation “cannot be adequately stated at this time due to a lack of adequate published reports,” Fayyazi & An, p. 58, and anterior C1-C2 lateral mass plates are “only indicated in selective cases and only when other methods of fixation cannot be used, Fayyazi & An, p. 59. Accordingly, there is a need in the art for new devices and methods for treating atlantoaxial injuries or pathological conditions.

### SUMMARY

In accordance with the principles of the present invention, as embodied and broadly described herein, new devices and methods are provided for treating atlantoaxial injuries or pathological conditions. These devices and methods may provide treatment while reducing one or more of the necessity of performing multiple operations, the likelihood of complications and post-operative discomfort, the number of days a patient will likely remain hospitalized after surgery, the risk of injury to important structures like nerve roots and blood vessels, and the potential for bone fixation instability.

According to an embodiment of the present invention, a plate for contacting at least two cervical vertebrae of a spine is provided. The plate may include a first portion configured to contact the first cervical vertebra of the spine, the first portion including a hole arranged on a longitudinal axis of the plate, and the hole being configured to receive a screw adapted to be screwed into the anterior tubercle of the first cervical vertebra. The plate may also include a second portion configured to contact at least one of the second and third cervical vertebrae of the spine, the second portion including a pair of holes arranged on an axis substantially perpendicular to the longitudinal axis of the plate, and each of the holes in the pair of holes being configured to receive a screw adapted to be screwed into the body of one of the second and third cervical vertebrae.

There is also provided a plate for contacting at least three cervical vertebrae of a spine. According to an embodiment of the present invention, the plate may include a first section configured to contact the anterior tubercle of the C1 vertebra of a spine, the first section including a hole arranged on a longitudinal axis of the plate. The plate may also include a second section configured to contact the body of any two or more of the C2, C3, and C4 vertebrae of the spine, the second section including a first pair of holes arranged on a first axis substantially perpendicular to the longitudinal axis of the plate and a second pair of holes arranged on a second axis substantially perpendicular to the longitudinal axis of the plate.

Further, there is also provided a surgical kit. According to an embodiment of the present invention, the kit may include a plate that itself may include a first portion configured to contact the C1 vertebra of a spine, the first portion including a hole configured to receive a screw adapted to be screwed into the anterior tubercle of the C1 vertebra. The plate may also include a second portion configured to contact at least one of the C2, C3, and C4 vertebrae of the spine, the second portion including at least one pair of holes configured to receive a screw adapted to be screwed into the body of the at least one of the C2, C3, and C4 vertebrae. The kit may further include at least one screw adapted to be screwed into the anterior tubercle of the C1 vertebra, the at least one screw having a thickness between about 1.7 mm and about 2.7 mm.

Furthermore, there is provided a method for surgically fixating at least two cervical bones. According to an embodiment of the present invention, the method may include surgically accessing the anterior surface of the first cervical vertebra of a spine and at least one of the second, third, and fourth cervical vertebrae of the spine. The method may also include placing a first portion of a plate including a hole onto a portion of the anterior tubercle of the first cervical vertebra and inserting a screw into the anterior tubercle of the first cervical vertebra through the hole in the first portion of the plate to secure the plate to the first cervical vertebra. Further, the method may also include placing a second portion of the plate including at least one pair of holes onto a portion of the body of at least one of the second, third, and fourth cervical vertebrae; and inserting a screw into the body of the at least one of the second, third, and fourth cervical vertebrae through each hole in the at least one pair of holes of the second portion of the plate.

Additional objects and embodiments of the present invention will be set forth in or flow from the description that follows, and may in part be obvious in view of the description of the present invention, or may be learned by practice of the present invention. The objects of the present invention will be realized and attained by means of the elements and combinations particularly pointed out in the appended claims.

It is to be understood that both the foregoing general description and the following detailed description are exemplary and explanatory only and are not restrictive of the invention, as described. Further features and/or variations may be provided in addition to those set forth herein. For example, the present invention may be directed to various combinations and subcombinations of several further features disclosed below in the detailed description.

### BRIEF DESCRIPTION OF THE DRAWINGS

The accompanying drawings, which are incorporated in and constitute a part of this specification, illustrate several embodiments of the invention and serve to explain certain features and principles of the invention. The drawings, however, are merely examples and are not limiting in any way of the scope of the invention, which scope shall be determined solely based on the appended claims, and a person of ordinary skill in the art will appreciate that countless variations from these non-limiting, exemplary representations are possible and readily flow from the description that follows.

FIG. 1A illustrates a top view of a plate having three holes for contacting at least two vertebrae according to an embodiment of the present invention.

FIG. 1B illustrates a cross-sectional view of a curved plate having three holes for contacting at least two vertebrae as in FIG. 1A, the plate being viewed along its longitudinal axis, according to another embodiment of the present invention.

FIG. 1C illustrates a perspective view of a curved plate having three holes for contacting at least two vertebrae as in FIG. 1A according to another embodiment of the present invention.

FIG. 1D illustrates a cross-sectional view of a flat plate having three holes for contacting at least two vertebrae as in FIG. 1A, the plate being viewed along an axis perpendicular to its longitudinal axis, according to another embodiment of the present invention.

FIG. 1E illustrates a cross-sectional view of a bent plate having three holes for contacting at least two vertebrae as in FIG. 1A, the plate being viewed along an axis perpendicular to its longitudinal axis, according to another embodiment of the present invention.

FIG. 1F illustrates a cross-sectional view of a curved plate having three holes for contacting at least two vertebrae as in FIG. 1A and a concave portion surrounding one of the holes, the plate being viewed along an axis perpendicular to its longitudinal axis, according to another embodiment of the present invention.

FIG. 2A illustrates a top view of a substantially triangular plate having three holes for contacting at least two vertebrae according to another embodiment of the present invention.

FIG. 2B illustrates a top view of a substantially triangular plate having a central perforation and three holes for contacting at least two vertebrae according to another embodiment of the present invention.

FIG. 2C illustrates a top view of a substantially “inverted T”-shaped plate having three holes for contacting at least two vertebrae according to another embodiment of the present invention.

FIG. 3A illustrates a top view of a plate having five holes for contacting at least three vertebrae according to another embodiment of the present invention.

FIG. 3B illustrates a top view of a plate having a central perforation and five holes for contacting at least three vertebrae according to another embodiment of the present invention.

FIG. 3C illustrates a cross-sectional view of a plate as illustrated in FIG. 3A or FIG. 3B, the plate having a concave portion surrounding one of the holes and being viewed along an axis perpendicular to its longitudinal axis, according to another embodiment of the present invention.

FIG. 4A illustrates a close-up view of a hole in a portion of a plate along with surrounding serrations or notches according to an embodiment of the present invention.

FIG. 4B illustrates a cross-sectional view of a portion of a plate in which a screw has been inserted and onto which a staple has been placed according to an embodiment of the present invention.

FIG. 4C illustrates a close-up view of a hole flanked by serrations in a portion of a plate in which a screw having notches on its surface has been inserted and aligned with the plate's serrations according to an embodiment of the present invention.

FIG. 5A illustrates a top view of a plate having three holes for contacting at least two vertebrae as in FIG. 1A along with screws with surface notches inserted into the holes and locking staples stapled onto the screws to lock them and the plate into position according to another embodiment of the present invention.

FIG. 5B illustrates a top view of a plate having a central perforation and five holes for contacting at least three vertebrae as in FIG. 3B along with screws with surface notches inserted into the holes and locking staples stapled onto the screws to lock them and the plate into position according to another embodiment of the present invention.

FIG. 6A illustrates a top view of a plate having three holes for contacting at least two vertebrae as in FIG. 1A along with gripping members according to another embodiment of the present invention.

FIG. 6B illustrates a top view of a plate having a central perforation and five holes for contacting at least three vertebrae as in FIG. 3B along with gripping members according to another embodiment of the present invention.

FIG. 7A illustrates a plate with five holes for contacting at least three vertebrae along with screws and side notches according to another embodiment of the present invention.

FIG. 7B illustrates a plate with two holes for contacting at least two vertebrae along with screws and side notches according to another embodiment of the present invention.

FIG. 7C illustrates a plate with five holes for contacting at least three vertebrae along with screws and multiple side notches according to another embodiment of the present invention.

FIG. 8 illustrates a spacer having a cross-section that varies from substantially circular to substantially elliptical inserted between two vertebrae linked together via a plate screwed into each of the vertebrae.

### EXEMPLARY EMBODIMENTS

Reference will now be made in detail to exemplary embodiments of the invention, examples of which are illustrated in the accompanying drawings. Wherever convenient, similar reference numbers will be used throughout the drawings to refer to the same or like parts. The implementations set forth in the following description do not represent all implementations consistent with the claimed invention. Instead, they are merely some examples of devices and methods consistent with the invention.

Fixation of C1 and C2, or of C1 and one or more of C2, C3, and C4, can be safely and efficaciously accomplished in, for example, a single surgical anterior procedure wherein damaged or diseased spinal tissue is initially excised to decompress the spine and wherein C1 and C2, or C1 and one or more of C2, C3, and C4, are subsequently fixated using a novel anterior procedure and novel cervical plate. Several illustrative embodiments of such a procedure and plate are discussed next.

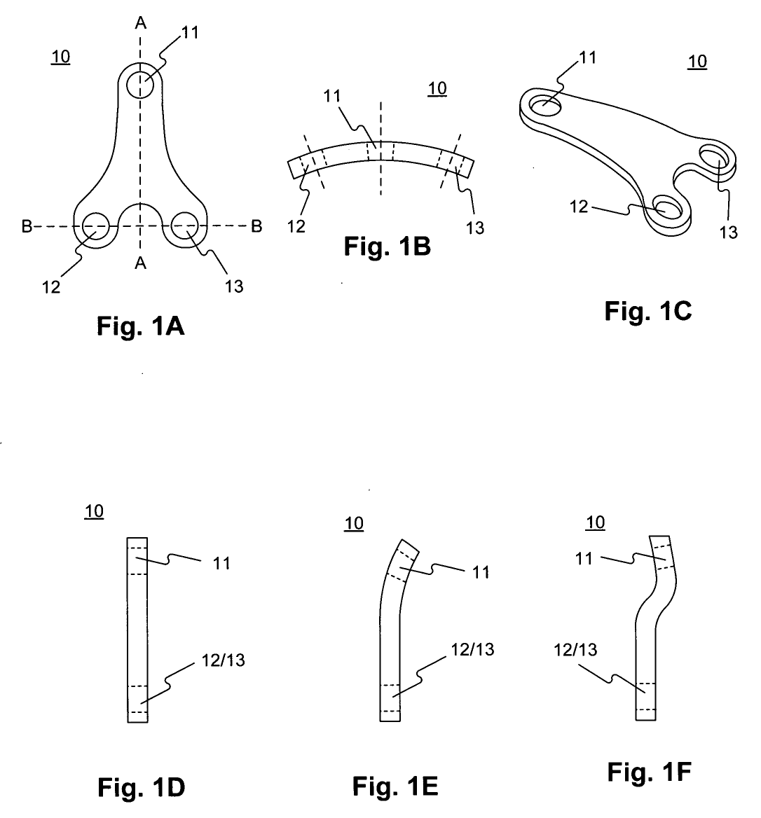

FIG. 1A illustrates a top view of a plate 10 for contacting at least two vertebrae according to an embodiment of the present invention. Plate 10 may include a first portion having a hole 11 arranged along a longitudinal axis A-A of the plate 10. Plate 10 may also include a second portion having two holes 12 and 13 arranged along an axis B-B substantially perpendicular to the longitudinal axis A-A of the plate 10. The second portion may have branching limbs extending on either sides of the longitudinal axis A-A so as to render the plate 10 substantially “Y”-shaped. The holes 12 and 13 of the second portion may be arranged so that there is one hole on each side of the longitudinal axis A-A. The holes 12 and 13 may be located on either side of the longitudinal axis A-A at a same or similar distance from the longitudinal axis A-A, though they may also be located at different distances from the longitudinal axis A-A. While plate 10 may have a substantially rounded contour, as illustrated in FIG. 1A, it may also have any other type of contour, including a contour with one or more right angles.

The first portion of plate 10 may be configured to contact the first cervical vertebra of the spine, i.e., the atlas or C1. More specifically, the hole 11 may be configured to receive a screw adapted to be screwed into the anterior tubercle of the first cervical vertebra. The screw may generally be thinner than screws conventionally used in subaxial plating systems and may have a thickness between about 1.7 mm and about 2.7 mm, and a length between about 6 mm and about 10 mm, or between about 6 mm and about 8 mm. The second portion of the plate 10 may be configured to contact one or more of the second, third, and fourth cervical vertebrae of the spine, i.e., the axis or C2, C3, and C4. The holes 12 and 13 may be configured to receive a screw adapted to be screwed into the body of the second, third, or fourth cervical vertebra. This screw may have a thickness between about 2.6 mm and about 3.2 mm, and a length between about 10 mm and about 20 mm, or between about 14 mm and about 18 mm. The holes 12 and 13 may be configured to receive a screw adapted to be screwed into the body of C2 or C3. Alternatively, the holes 12 and 13 may be configured to receive a screw adapted to be screwed into the body of C2. Any type of cervical plating system screws known in the art may be used with any of the plates discussed herein.

The holes 11-13 may have any shape or attributes used in any known cervical plating system. For example, the holes 11-13 may be substantially cylindrical or may have a diameter that varies smoothly and/or abruptly along their length, and they may have a diameter at their narrowest point that is between about 1 mm and about 10 mm. Alternatively, the hole 11 may have a diameter at its narrowest point that is between about 1.7 mm and about 4.7 mm, and the holes 12 and 13 may have a diameter at their narrowest point that is between about 2.6 mm and about 4.6 mm. Alternatively, the hole 11 may have a diameter at its narrowest point that is between about 1.7 mm and about 2.7 mm, and the holes 12 and 13 may have a diameter at their narrowest point that is between about 2.6 mm and about 3.2 mm. These ranges for the dimensions of the screws and holes may allow enhanced stability of an anterior plate contacting the atlas and C2 or C3.

The distance between the center of the hole 11 and the axis B-B may vary according to which vertebra the second portion of the plate 10 will be contacting and may correspond to any anatomically possible distances separating C1 and any one of C2, C3, and C4. The distance between the center of the hole 11 and the axis B-B may be between about 10 mm and about 40 mm. The centers of the holes 12 and 13 may be separated by a distance between about 2.7 mm and about 15 mm. Alternatively, the centers of the holes 12 and 13 may be separated by a distance between about 2.7 mm and about 10 mm. These relatively short distances between the holes 12 and 13 may allow enhanced stability of an anterior plate contacting the atlas and the axis and minimize risks to the patient during surgery by ensuring that the operation will be conducted close to the midline, thus reducing the risks of injury to nerve roots and blood vessels located laterally.

Plate 10 may have one or more curvatures with respect to one or more axes. For example, FIG. 1B illustrates a possible cross-sectional view of plate 10, viewed along its longitudinal axis, where plate 10 has a curvature corresponding to anatomically possible anterior surface curvatures of the second, third, or fourth cervical vertebra. Such a curvature may allow a better contact between the second portion of the plate 10 and the second, third, or fourth cervical vertebra, as the case may be, along with improved stability of the plate 10. A plate 10 as illustrated in FIG. 1B may have a radius of curvature between about 10 mm and about 30 mm. Alternatively, that radius of curvature may be between about 15 mm and about 25 mm. Along the same lines, FIG. 1C illustrates a perspective view of a plate 10 with a profile curved lengthwise to allow the plate, for example, to connect C1 and C3 while passing over the C2 anterior protuberance that might otherwise prevent a flat plate from connecting C1 and C3 in some patients. A plate 10 as illustrated in FIG. 1C may have a radius of curvature between about 5 cm and about 25 cm. Further, plate 10 may have a bi-axial curvature incorporating the features of both FIGS. 1B and 1C.

Many other configurations are, of course, possible. For example, FIG. 1D illustrates a cross-sectional view of a plate 10 that is flat, which may be used, for example, when fixating C1 and C2 in some patients or when fixating C1 and C3 in patients where the C2 anterior protuberance is absent or otherwise not problematic. FIG. 1E illustrates a cross-sectional view of a plate 10 viewed sideways where the plate is bent so as to cause the first and second portions of the plate 10 to be inclined relative to each other. The first and section portions of the plate 10 may be inclined relative to each other at an angle between about 135 degrees and about 179 degrees. Alternatively, they may be inclined at an angle between about 145 degrees and about 165 degrees. Finally, FIG. 1F illustrates a cross-sectional view of a cervical plate viewed sideways where the plate has a concave portion surrounding the hole of the first section, which may be useful to ensure as stable a fit as possible between the first portion of the plate and the anterior tubercle of the atlas.

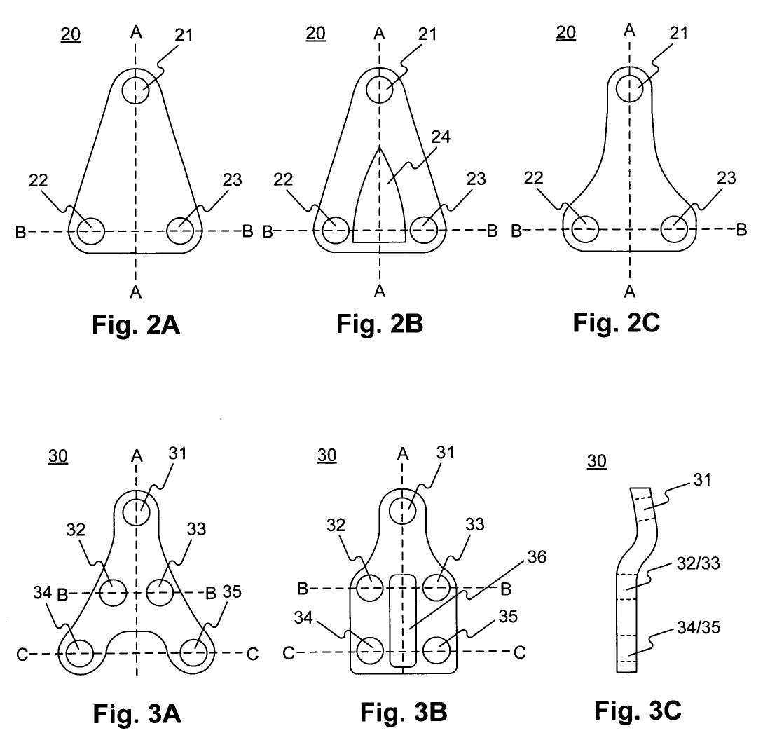

FIG. 2A illustrates a top view of a substantially triangular plate 20 for contacting at least two vertebrae according to another embodiment of the present invention. Plate 20 may include a first portion having a hole 21 arranged along a longitudinal axis A-A of the plate 20. Plate 20 may also include a second portion having two holes 22 and 23 arranged along an axis B-B substantially perpendicular to the longitudinal axis A-A of the plate 20. Plate 20 may be shaped as a generally isosceles triangle, with the hole 21 of the first portion being arranged in the corner of the triangle have the smallest angle and the holes 22 and 23 being arranged in the other two corners of the triangle. While plate 20 may have a substantially rounded contour, as illustrated in FIG. 2A, it may also have any other type of contour.

The first portion of plate 20 may be configured to contact C1, and the hole 21 may be configured to receive a screw adapted to be screwed into the anterior tubercle of C1. The screw may have a thickness between about 1.7 mm and about 2.7 mm. The second portion of the plate 20 may be configured to contact one or more of C2, C3, and C4, and the holes 22 and 23 may be configured to receive a screw adapted to be screwed into the body of the second, third, or fourth cervical vertebra. This screw may have a thickness between about 2.6 mm and about 3.2 mm. The holes 22 and 23 may be configured to receive a screw adapted to be screwed into the body of C2. Among many possible variations of FIG. 2A, FIG. 2B illustrates a top view of a substantially triangular plate 20 having a central perforation 24 and three holes 21, 22, and 23 for contacting at least two vertebrae according to another embodiment of the present invention, and FIG. 2C illustrates a top view of a substantially “inverted T”-shaped plate 20 having three holes 21, 22, and 23 for contacting at least two vertebrae according to another embodiment of the present invention. The dimensions, locations, and any other properties of the holes discussed above regarding the holes 11, 12, and 13 of FIGS. 1A-1F also apply to the holes 21, 22, and 23 of plate 20. The dimensions and any other properties of the screws discussed above with respect to FIGS. 1A-1F also apply to the screws for plate 20. Further, plate 20 may also be flat or characterized with various curvatures, as discussed above regarding FIGS. 1A-1F.

FIG. 3A illustrates a top view of a plate 30 for contacting at least three vertebrae according to another embodiment of the present invention. Plate 30 may include a first portion having a hole 31 arranged along a longitudinal axis A-A of the plate 30. Plate 30 may also include a second portion having four holes 32-35, the holes 32 and 33 being arranged along an axis B-B substantially perpendicular to the longitudinal axis A-A of the plate 30, and the holes 34 and 35 being arranged along another axis C-C substantially perpendicular to the longitudinal axis A-A of the plate 30. Plate 30 may be “Y”-shaped, with the holes 32 and 33 being located around the branching area of the “Y” and one of the holes 34 and 35 being located in each of the branches of the “Y” as illustrated in FIG. 3A. The holes 32-35 may be arranged so that there are two holes on each side of the longitudinal axis A-A, and the holes 34 and 35 may be arranged farther from the longitudinal axis A-A than the holes 32 and 33. Alternatively, the holes 34 and 35 may be arranged at a same or similar distance from the longitudinal axis A-A than the holes 32 and 33, as illustrated in FIG. 3B, in which case the plate 30 may include a perforation 36 that may be useful for screw and plate locking purposes, as discussed below. While plate 30 may have a substantially rounded contour, as illustrated in FIG. 3A, it may also have any other type of contour.

The first portion of plate 30 may be configured to contact C1, and the hole 31 may be configured to receive a screw adapted to be screwed into the anterior tubercle of C1. The screw may have a thickness between about 1.7 mm and about 2.7 mm. The second portion of the plate 30 may be configured to contact either C2 and C3, C2 and C4, or C3 and C4, and the holes 32-35 may be configured to receive a screw adapted to be screwed into the body of C2, C3, or C4. This screw may have a thickness between about 2.6 mm and about 3.2 mm. The second portion of the plate 30 may also be configured to contact C2, C3, and C4, thus constituting a C1-C2-C3-C4 plate, which may be useful in certain Hangman's fracture cases. The holes 32 and 33 may be configured to receive a screw adapted to be screwed into the body of C2 and the holes 34 and 35 may be configured to receive a screw adapted to be screwed into the body of C3. The dimensions, locations, and any other properties of the holes discussed above regarding the holes 11, 12, and 13 of FIGS. 1A-1F also apply to the holes 31-35 of plate 30. The distance between the center of the hole 31 and the axes B-B and C-C may vary according to which vertebrae the second portion of the plate 30 will be contacting and may correspond to any anatomically possible distances separating C1 and any two of C2, C3, and C4. The distance between the center of the hole 31 and the axis C-C may be between about 10 mm and about 50 mm. The dimensions and any other properties of the screws discussed above with respect to FIGS. 1A-1F also apply to the screws for plate 30. Further, plate 30 may be flat or characterized with various curvatures, as discussed above regarding FIGS. 1A-1F. An example of a possible curvature of plate 30, including a concave area surrounding the hole 31, is represented in FIG. 3C.

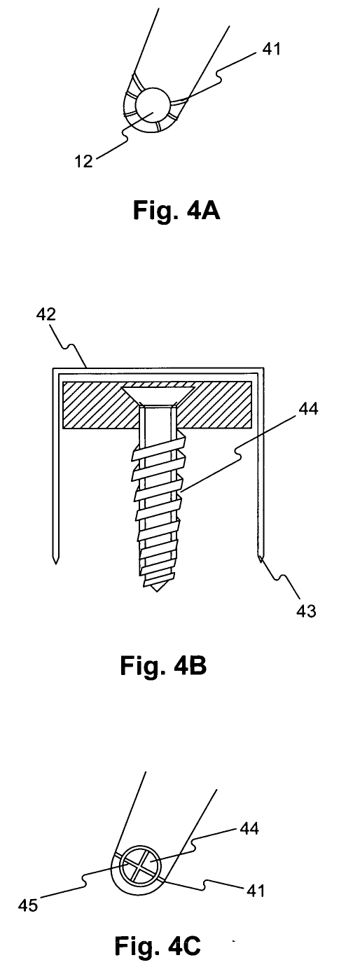

Any of these embodiments may benefit from the addition of one or more screw and/or plate locking systems conventionally used in the art, such as locking screws and their associated holes, to further enhance plate stability. Alternatively, any of these embodiments may benefit from the addition of a novel staple-based locking system, which is discussed next. As illustrated in FIG. 4A, which shows a close-up view of a hole 12 in a portion of a plate, the hole 12, or any hole in any plate according to embodiments of the present invention, may be surrounded with one or more serrations or notches 41 configured to receive part of a locking staple. As illustrated in FIG. 4B, which shows a cross-sectional view of a portion of a plate in which a screw 44 has been inserted, a staple 42 having sharp endings 43 may be placed onto the plate and the screw to lock them in position. Screw 44 may be inserted so that its surface is flush with the surface of the plate. Alternatively, it may be inserted so that its surface is depressed relative to that of the plate. As illustrated in FIG. 4C, not only may the hole be flanked by notches 41, but the surface of the screw 44 may also be covered with one or more notches 45 so that a staple can very effectively lock the plate and screw in position by being placed partially or fully into the notches 41 and 45, when suitably aligned. The notches 41 and 45 may have a width between about 0.2 mm and about 2 mm, and a depth between about 0.1 mm and about 1 mm.

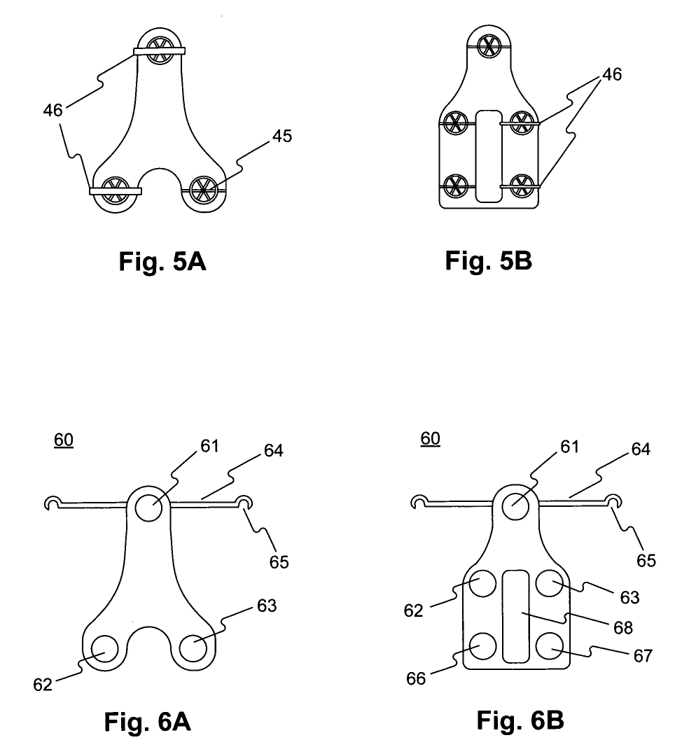

To better appreciate the features of this novel staple locking system, FIG. 5A illustrates a top view of a plate for contacting at least two vertebrae as in FIG. 1A along with screws with surface notches 45 inserted into the holes of the plate and locking staples 46 stapled onto the screws to lock them and the plate into position. Along the same lines, FIG. 5B illustrates a top view of a plate having a central perforation and five holes for contacting at least three vertebrae as in FIG. 3B along with screws with surface notches inserted into the holes and locking staples 46 stapled onto the screws to lock them and the plate into position. While FIGS. 5A and 5B show two staples 46, any one or more of the holes of a plate may be locked with such staples. The staples 46 may have a width between about 0.2 mm and about 2 mm, and a thickness between about 0.1 mm and about 1 mm to fit within the notches 45. However, the staples 46 may also be wider and thicker than the notches 45 and only partially be inserted into the notches, such as via a protruding segment under the staples 46 (not shown) configured to be inserted into the notches 45. The staples 46 may be made of any material conventionally used in the field of implantable medical devices. The staples 46 may include one or more biocompatible materials, such as titanium dioxide, Teflon, etc. The staples 46 may also include or be made of one or more biodegradable materials, such as polyglycolic acid. According to another embodiment of the present invention, a plate for contacting C1 and one or more other vertebrae may be stabilized even further by using members configured to wrap around and grab the C1 anterior arch. FIG. 6A illustrates a top view of a plate 60 having three holes 61-63 for contacting at least two vertebrae as in FIG. 1A along with a pair of gripping members 64 and hooks or claws 65 arranged on the sides of the plate near the first portion of the plate and configured to grip or clasp onto the C1 anterior arch. Similarly, FIG. 6B illustrates a top view of a plate 60 having a central perforation 68 and five holes 61-63, 66, and 67 for contacting at least three vertebrae as in FIG. 3B along with a pair of gripping members 64 and hooks or claws 65 arranged on the sides of the plate near the first portion of the plate. The gripping members may have a length between about 5 mm and about 20 mm.

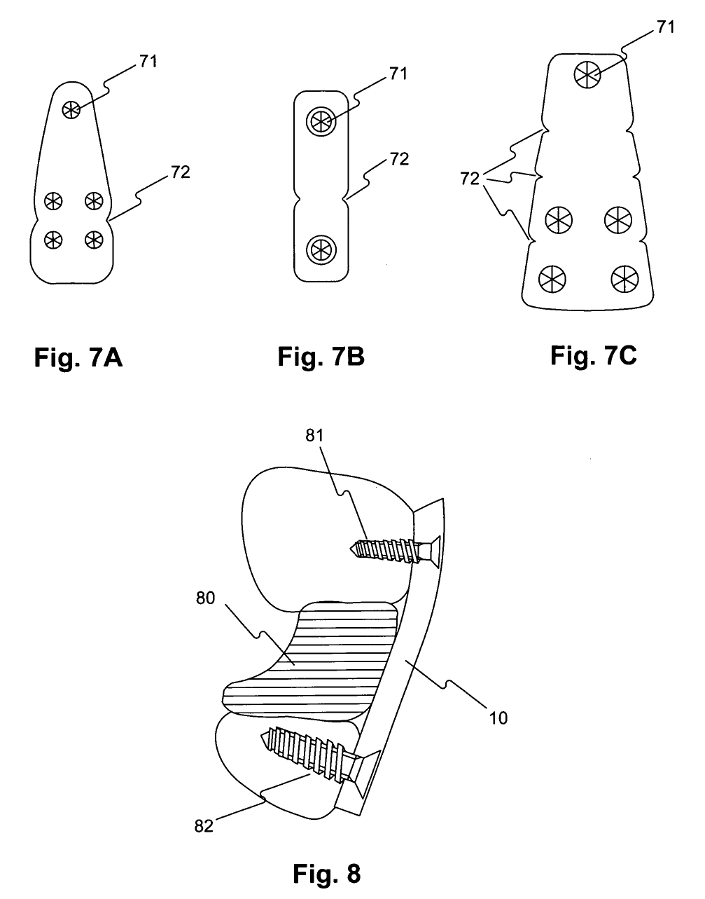

Numerous additional configurations are possible. For example, FIG. 7A illustrates a plate with five holes for contacting at least three vertebrae along with screws 71 and side notches 72 according to another embodiment of the present invention, FIG. 7B illustrates a plate with two holes for contacting at least two vertebrae along with screws 71 and side notches 72 according to another embodiment of the present invention, and FIG. 7C illustrates a plate with five holes for contacting at least three vertebrae along with screws 71 and multiple side notches 72 according to another embodiment of the present invention. In these cases, larger staples may be stapled through the side notches to lock the plate into position.

The various aforementioned plates may be used in combination with a spacer. For example, as illustrated in FIG. 8, a spacer 80 having a cross-section that varies from substantially circular to substantially elliptical may be inserted between two vertebrae linked together via a plate screwed into each of the vertebrae according to an embodiment of the present invention. This may be very useful, for example, to treat rheumatoid arthritis, an increasingly important concern given the aging of population, which may require the excision of the odontoid process and pannus tissue and the subsequent fixation of C1 to C2 or C3 using a plate and a spacer to fill the gap resulting from the excision. The spacer may be made of any material conventionally used in the field of implantable medical devices. The spacer may include one or more biocompatible materials, such as titanium. The spacer may also include one or more biodegradable materials, such as polyglycolic acid. Screw 81 may be thinner and shorter than screw 82. Screws 81 and 82, and any other screws discussed herein, may be of any type of screws known in the art as suitable to be screwed into any bone. Moreover, to stabilize the plate and the spacer even further, one or more staples may be inserted over the plate and into the spacer so that the plate and the space will behave as a single unit to prevent sideways slipping.

The various plates discussed above may have a thickness that is less than the thickness of other plates conventionally used in subaxial plating systems, though conventional thicknesses are also possible. In particular, the various plates discussed above may have a thickness between about 0.5 mm and about 2 mm. Alternatively, they may have a thickness between about 1 mm and about 1.8 mm. Further, those plates may be made of any one or more materials conventionally used in subaxial plating systems, including titanium. Those plates may also be made of one or more biocompatible materials. Alternatively, those plates can be made of one or more biodegradable materials.

According to another embodiment of the invention, any of the aforementioned devices may conveniently be assembled into one or more surgical kits. A surgical kit may include any of the foregoing plates or variations thereof. For example, it may include a plate having a first portion configured to contact C1 and including a hole configured to receive a screw adapted to be screwed into the anterior tubercle of C1, which plate may also have a second portion configured to contact at least one of C2, C3, and C4 and may also include at least one pair of holes configured to receive a screw adapted to be screwed into the body of the at least one of C2, C3, and C4. The kit may also contain at least one screw adapted to be screwed into the anterior tubercle of the C1 vertebra. The screw may have a thickness between about 1.7 mm and about 2.7 mm, and may also have a length between about 6 mm and about 10 mm, or between about 6 mm and about 8 mm. The surgical kit may also include at least one pair of screws adapted to be screwed into the body of the at least one of C2, C3, and C4. These screws may have a thickness between about 2.7 mm and about 3.1 mm, and may also have a length between about 10 mm and about 20 mm, or between about 14 mm and about 18 mm. The surgical kit may include various other accessories. In particular, it may include one or more spacers configured to fill a gap between the anterior tubercle of C1 and one or more of C2, C3, and C4 and having a cross-section that varies gradually from substantially circular, at a first extremity of the spacer, to substantially elliptical, at a second extremity of the spacer. All other plate features discussed above may also be reflected in such a kit. For instance, any plate in the kit may have serrations extending from a periphery of each of the holes in the plate to receive a locking staple, and any screws in the kit may have similar serrations. Then, the kit may include a set of locking staples configured to be stapled into the serrations present in the screws and plate to lock the screws and the plate into position.

According to another embodiment of the present invention, any of the aforementioned devices may conveniently be implanted into a patient to fixate at least two cervical bones using the following method. First, the method may include surgically accessing the anterior surface of the first cervical vertebra of a spine and at least one of the second, third, and fourth cervical vertebrae of the spine. This may be performed anteriorly rather than posteriorly, and may be performed closely to the midline of the spine to avoid risks to important nerve roots and blood vessels located more laterally. Second, the method may include placing a first portion of a plate including a hole onto a portion of the anterior tubercle of the first cervical vertebra. Third, the method may include inserting a screw into the anterior tubercle of the first cervical vertebra through the hole in the first portion of the plate to secure the plate to the first cervical vertebra. Fourth, the method may include placing a second portion of the plate including at least one pair of holes onto a portion of the body of at least one of the second, third, and fourth cervical vertebrae. Finally, the method may include inserting a screw into the body of the at least one of the second, third, and fourth cervical vertebrae through each hole in the at least one pair of holes of the second portion of the plate.

As a person of ordinary skill in the art would appreciate, this exemplary procedure may be varied, modified, and supplemented in numerous ways, including changing the order of its procedural elements, without departing from the scope of the present invention. Inserting a screw into the anterior tubercle of the first cervical vertebra may include inserting a screw having a thickness between about 1.7 mm and about 2.7 mm, and may also include inserting a screw having a length between about 6 mm and about 8 mm. Inserting a screw into the body of the at least one of the second, third, and fourth cervical vertebrae may include inserting a screw having a thickness between about 2.7 mm and about 3.1 mm through each hole in the at least one pair of holes of the second portion of the plate, and may also include inserting a screw having a length between about 14 mm and about 18 mm through each hole in the at least one pair of holes of the second portion of the plate. The method may further include placing a plate having at least two gripping members configured to grip part of the anterior arch of the first cervical vertebra, and affixing the gripping members to the anterior arch of the first cervical vertebra to grip part of the anterior arch of the first cervical vertebra. It may also include placing a plate having serrations flanking each of the holes in the plate and being configured to receive at least one locking staple, and after having fixed the plate with screws also having on their surface serrations configured to receive at least one locking staple, inserting a locking staple over each of the screws and into the cervical vertebra in which that screw has been screwed such that each locking staple rests into the serrations of the plate and screws without protruding from the plate to lock the screws into position. Finally, the method may of course include placing a plate having any of the other characteristics discussed above such as, for example, a plate having a thickness between about 0.5 mm and about 2 mm, a plate made of one or more biodegradable materials, and a plate where the distance between the centers of the holes in the pair holes is between about 2.7 mm and about 15 mm.

The various exemplary embodiments of the present invention set forth above allow the treatment of atlantoaxial injuries or pathological conditions while reducing one or more of the necessity of performing multiple operations, the likelihood of complications and post-operative discomfort, the number of days a patient will likely remain hospitalized after surgery, the risk of injury to important structures like nerve roots and blood vessels, and the potential for bone fixation instability. These various exemplary embodiments, along with the many other embodiments of the invention, may thus substantially benefit patients who may suffer from various conditions including atlantoaxial injuries that may require bone removal and subsequent fixation, atlantoaxial dislocations resulting from “Hangman's fractures” that may require surgery to reduce the extent of any dislocation, and the destruction of the odontoid process and/or pannus tissue that can result from rheumatoid arthritis or from atlantoaxial tumors.

Other embodiments of the invention will be apparent to those skilled in the art from consideration of the specification and practice of the invention disclosed herein. In particular, any sub-range contained in any range disclosed in this specification is also contemplated as a possible alternative to the broader disclosed range containing that sub-range. Finally, it is intended that the specification and examples be considered as exemplary only, with a true scope and spirit of the invention being indicated by the following claims.

1. A plate for contacting at least two cervical vertebrae of a spine, comprising:
a first portion configured to contact the first cervical vertebra of the spine, the first portion including a hole arranged on a longitudinal axis of the plate, the hole being configured to receive a screw adapted to be screwed into the anterior tubercle of the first cervical vertebra; and
a second portion configured to contact at least one of the second and third cervical vertebrae of the spine, the second portion including a pair of holes arranged on an axis substantially perpendicular to the longitudinal axis of the plate, each of the holes in the pair of holes being configured to receive a screw adapted to be screwed into the body of one of the second and third cervical vertebrae.

2. The plate of claim 1, wherein the distance between the center of the hole in the first portion and the axis substantially perpendicular to the longitudinal axis of the plate is between about 10 mm and about 40 mm.

3. The plate of claim 1, wherein the distance between the centers of the holes in the pair of holes is between about 2.7 mm and about 15 mm.

4. The plate of claim 1, wherein the distance between the centers of the holes in the pair of holes is between about 2.7 mm and about 10 mm.

5. The plate of claim 1, wherein the hole in the first portion has a diameter at its narrowest point that is between about 1.7 mm and about 2.7 mm.

6. The plate of claim 1, wherein each of the holes in the pair of holes in the second portion has a diameter at its narrowest point that is between about 2.6 mm and about 3.2 mm.

7. The plate of claim 5, wherein each of the holes in the pair of holes in the second portion has a diameter at its narrowest point that is between about 2.6 mm and about 3.2 mm.

8. The plate of claim 1, wherein the plate is Y-shaped such that the second portion comprises two limbs extending on either sides of the longitudinal axis, each of the two limbs having one of the holes of the pair of holes.

9. The plate of claim 1, wherein the second portion of the plate has a curvature such that a radius of curvature along the axis substantially perpendicular to the longitudinal axis of the plate is between about 15 mm and about 25 mm.

10. The plate of claim 1, wherein the plate has a curvature such that a radius of curvature along the longitudinal axis of the plate is between about 5 cm and about 25 cm.

11. The plate of claim 1, wherein the plate is substantially flat.

12. The plate of claim 1, wherein the plate is bent along its longitudinal axis such that an angle between the first and second portions of the plate is between about 135 degrees and about 179 degrees.

13. The plate of claim 1, wherein the first portion of the plate includes a concave portion configured to match a convexity of the anterior tubercle of the first cervical vertebra, the concave portion including the hole of the first portion.

14. The plate of claim 1, wherein the plate has a substantially isosceles triangular shape, the first portion of the plate corresponding to the corner of the plate having the smallest angle, and each of the holes in the pair of holes being arranged in the remaining corners of the plate.

15. The plate of claim 14, further comprising a perforated portion located around a central area of the plate.

16. The plate of claim 1, wherein the plate is substantially shaped as an inverted T with rounded contour.

17. The plate of claim 1, further comprising at least two members extending from the first portion of the plate, each of the at least two members comprising a gripping portion configured to clasp part of the anterior arch of the first cervical vertebra.

18. The plate of claim 1, wherein each of the holes in the plate is flanked by one or more notches having a width between about 0.2 mm and about 2 mm, and a depth between about 0.1 mm and about 1 mm.

19. The plate of claim 1, wherein the plate has a thickness between about 0.5 mm and about 2 mm.

20. The plate of claim 1, where the plate is made of one or more biodegradable materials.

21. A plate for contacting at least three cervical vertebrae of a spine, comprising:
a first section configured to contact the anterior tubercle of the C1 vertebra of a spine, the first section including a hole arranged on a longitudinal axis of the plate; and
a second section configured to contact the body of any two or more of the C2, C3, and C4 vertebrae of the spine, the second section including a first pair of holes arranged on a first axis substantially perpendicular to the longitudinal axis of the plate and a second pair of holes arranged on a second axis substantially perpendicular to the longitudinal axis of the plate.

22. The plate of claim 21, wherein the distance between the centers of the holes in each of the first and second pairs of holes is between about 2.7 mm and about 15 mm.

23. The plate of claim 21, wherein the hole in the first section has a diameter at its narrowest point that is between about 1.7 mm and about 2.7 mm.

24. The plate of claim 21, wherein each of the holes in the first and second pairs of holes has a diameter at its narrowest point that is between about 2.6 mm and about 3.2 mm.

25. The plate of claim 21, wherein the second section of the plate has a curvature such that a radius of curvature along the first and second axes substantially perpendicular to the longitudinal axis of the plate is between about 15 mm and about 25 mm.

26. The plate of claim 21, wherein the first section of the plate includes a concave portion configured to match a convexity of the anterior tubercle of the C1 vertebra, the concave portion including the hole of the first section.

27. The plate of claim 21, further comprising a perforated portion located around a central area of the plate.

28. The plate of claim 21, further comprising at least two members extending from the first section of the plate, each of the at least two members comprising a gripping portion configured to clasp part of the anterior arch of the C1 vertebra.

29. The plate of claim 21, wherein each of the holes in the plate is flanked by one or more serrations extending from a periphery of that hole, the serrations being configured to receive a staple configured to lock any screw screwed through that hole, and the serrations having a width between about 0.2 mm and about 2 mm, and a depth between about 0.1 mm and about 1 mm.

30. The plate of claim 21, wherein the plate has a thickness between about 0.5 mm and about 2 mm.

31. A surgical kit, comprising:
a plate comprising:
a first portion configured to contact the C1 vertebra of a spine, the first portion including a hole configured to receive a screw adapted to be screwed into the anterior tubercle of the C1 vertebra; and
a second portion configured to contact at least one of the C2, C3, and C4 vertebrae of the spine, the second portion including at least one pair of holes configured to receive a screw adapted to be screwed into the body of the at least one of the C2, C3, and C4 vertebrae; and
at least one screw adapted to be screwed into the anterior tubercle of the C1 vertebra, the at least one screw having a thickness between about 1.7 mm and about 2.7 mm.

32. The surgical kit of claim 31, wherein the at least one screw has a length between about 6 mm and about 10 mm.

33. The surgical kit of claim 31, further comprising at least one pair of screws adapted to be screwed into the body of the at least one of the C2, C3, and C4 vertebrae, the at least one pair of screws having a thickness between about 2.7 mm and about 3.1 mm.

34. The surgical kit of claim 33, wherein the at least one pair of screws adapted to be screwed into the body of the at least one of the C2, C3, and C4 vertebrae have a length between about 14 mm and about 18 mm.

35. The surgical kit of claim 31, further comprising a spacer configured to fill a gap between the anterior tubercle of the C1 vertebra and one or more of the C2, C3, and C4 vertebrae, the spacer having a cross-section that varies gradually from substantially circular, at a first extremity of the spacer, to substantially elliptical, at a second extremity of the spacer.

36. The surgical kit of claim 31, wherein the plate further includes serrations extending from a periphery of each of the holes in the plate, the serrations being configured to receive a locking staple.

37. The surgical kit of claim 36, wherein the serrations have a width between about 0.2 mm and about 2 mm, and a depth between about 0.1 mm and about 1 mm.

38. The surgical kit of claim 37, further comprising at least one pair of screws adapted to be screwed into the body of the at least one of the C2, C3, and C4 vertebrae, wherein all the screws in the surgical kit include on their surface serrations having a width between about 0.2 mm and about 2 mm, and a depth between about 0.1 mm and about 1 mm.

39. The surgical kit of claim 38, further comprising a set of locking staples configured to be stapled into the serrations present in the screws and plate to lock the screws and the plate into position.

40. The surgical kit of claim 31, wherein the plate further includes at least two members extending from the plate and each comprising a gripping portion configured grip part of the anterior arch of the C1 vertebra.

41. A method for surgically fixating at least two cervical bones, comprising:
surgically accessing the anterior surface of the first cervical vertebra of a spine and at least one of the second, third, and fourth cervical vertebrae of the spine;
placing a first portion of a plate including a hole onto a portion of the anterior tubercle of the first cervical vertebra;
inserting a screw into the anterior tubercle of the first cervical vertebra through the hole in the first portion of the plate to secure the plate to the first cervical vertebra;
placing a second portion of the plate including at least one pair of holes onto a portion of the body of at least one of the second, third, and fourth cervical vertebrae; and
inserting a screw into the body of the at least one of the second, third, and fourth cervical vertebrae through each hole in the at least one pair of holes of the second portion of the plate.

42. The method of claim 41, wherein inserting a screw into the anterior tubercle of the first cervical vertebra includes inserting a screw having a thickness between about 1.7 mm and about 2.7 mm.

43. The method of claim 41, wherein inserting a screw into the anterior tubercle of the first cervical vertebra includes inserting a screw having a length between about 6 mm and about 10 mm.

44. The method of claim 41, wherein inserting a screw into the body of the at least one of the second, third, and fourth cervical vertebrae includes inserting a screw having a thickness between about 2.7 mm and about 3.1 mm through each hole in the at least one pair of holes of the second portion of the plate.

45. The method of claim 41, wherein inserting a screw into the body of the at least one of the second, third, and fourth cervical vertebrae includes inserting a screw having a length between about 14 mm and about 18 mm through each hole in the at least one pair of holes of the second portion of the plate.

46. The method of claim 41, wherein placing a first portion of the plate onto a portion of the anterior tubercle of the first cervical vertebra includes placing a plate having at least two gripping members configured to grip part of the anterior arch of the first cervical vertebra; and further comprising
affixing the gripping members to the anterior arch of the first cervical vertebra to grip part of the anterior arch of the first cervical vertebra.

47. The method of claim 41, wherein placing a first portion of the plate onto a portion of the anterior tubercle of the first cervical vertebra includes placing a plate having serrations flanking each of the holes in the plate and being configured to receive at least one locking staple; and further comprising,
after having fixed the plate with screws also having on their surface serrations configured to receive at least one locking staple, inserting a locking staple over each of the screws and into the cervical vertebra in which that screw has been screwed such that each locking staple rests into the serrations of the plate and screws without protruding from the plate to lock the screws into position.

48. The method of claim 41, wherein placing a first portion of the plate onto a portion of the anterior tubercle of the first cervical vertebra includes placing a plate having a thickness between about 0.5 mm and about 2 mm.

49. The method of claim 41, wherein placing a first portion of the plate onto a portion of the anterior tubercle of the first cervical vertebra includes placing a plate made of one or more biodegradable materials.

50. The method of claim 41, wherein placing a second portion of the plate including at least one pair of holes onto a portion of the body of at least one of the second, third, and fourth cervical vertebrae includes placing a plate where the distance between the centers of the holes in the pair holes is between about 2.7 mm and about 15 mm.
